(19) United States
(12) Patent Application Publication
(10) Pub. No.: US 2005/0232866 A1
(43) Pub. Date: Oct. 20, 2005

(54) Biochemically-activated contrast agents for magnetic resonance imaging

(75) Inventors: Marco Melchior, San Diego, CA (US); Ying You, San Diego, CA (US); Serguei Calugaru, San Diego, CA (US); William C. Dow, La Jolla, CA (US)

Correspondence Address: MORGAN, LEWIS & BOCKIUS LLP (SF), 2 PALO ALTO SQUARE, PALO ALTO, CA, 94306, US

(73) Assignee: Alerion Biomedical Inc., San Diego, CA (US)

(21) Appl. No.: 11/090,644

(22) Filed: Mar. 25, 2005

Related U.S. Application Data
(60) Provisional application No. 60/556,839, filed on Mar. 26, 2004.

(51) Int. Cl. A61K 49/00

(52) U.S. Cl. 424/9.363; 424/9.364

(57) ABSTRACT

The present invention provides a class of biochemically-activated MRI contrast agents. The agents include a paramagnetic chelate attached via a linker arm to a relaxivity-modulating group. When the relaxivity-modulating group is removed or otherwise interacts with a targeted substance, the agents are activated by various mechanisms. The activation is detectable by an increase or decrease in the relaxivity of the metal chelate. The agents are useful in the detection of biochemical parameters in cells, tissues and subjects. Exemplary biochemical parameters are markers that are associated with disease states.

### CROSS-REFERENCE TO RELATED APPLICATIONS

This is a non-provisional filing of U.S. Provisional Patent Application No. 60/556,839, filed on Mar. 26, 2004, the disclosure of which is incorporated herein by reference in its entirety for all purposes.

### BACKGROUND OF THE INVENTION

Magnetic resonance imaging (MRI) is a diagnostic and research procedure that uses a large, high-strength magnet and radio-frequency signals to produce images. The most abundant molecular species in biological tissues is water. The quantum mechanical “spin” of the water proton nuclei is atomic characteristic that ultimately gives rise to the signal in all imaging experiments. In MRI the sample to be imaged is placed in a strong static magnetic field (1-12 Tesla) and the spins are excited with a pulse of radio frequency (RF) radiation to produce a net magnetization in the sample. Various magnetic field gradients and other RF pulses then act on the spins to code spatial information into the recorded signals. MRI is able to generate structural information in three dimensions in relatively short time spans.

Magnetic resonance (MR) images are typically displayed on a gray scale with black the lowest and white the highest measured intensity (I). This measured intensity is determined from the formula I=C*M, where C is the concentration of spins (in this case water concentration) and M is a measure of the magnetization present at time of the measurement. Although variations in water concentration (C) can give rise to contrast in MR images, it is the strong dependence of the rate of change of M on local environment that is the major source of image intensity variation in MRI. Two characteristic relaxation times are involved. T₁ is defined as the longitudinal relaxation time or spin lattice relaxation time (1/T₁ is a rate constant). T₂ is known as the transverse relaxation time or spin-spin mechanism; spin-spin interactions are one of several contributions to T₂ (1/T₂ is also a rate constant). T₁ and T₂ have reciprocal effects on image intensity, with image intensity increased by either shortening the T₁ or lengthening the T₂.

In order to increase the signal-to-noise ratio (SNR) a typical MR imaging scan (RF and gradient pulse sequence and data acquisition) is repeated at a constant rate a predetermined number of times and the data acquired are averaged. The signal amplitude recorded for any given scan is proportional to the number of spins that have decayed back to equilibrium since the previous scan. Thus, regions with rapidly decaying spins (i.e. short T₁ values) will recover all of their signal amplitude between successive scans. Their measured intensities in the final image will accurately reflect the spin density (i.e. water content). Regions with long T₁ values compared to the time between scans will progressively lose signal until a steady state condition is reached and will appear as darker regions in the final image. In extreme situations the linewidth is so large that the signal is indistinguishable from background noise. In clinical imaging, water relaxation characteristics vary from tissue to tissue, providing the contrast that allows the discrimination of tissue types. Moreover, the MRI experiment can be set up so that regions of the sample with short T₁ values and/or long T₂ values are preferentially enhanced-so called T₁-weighted and T₂-weighted imaging protocols.

A rapidly growing body of literature demonstrates the clinical effectiveness of paramagnetic contrast agents; currently there are at least eight different contrast agents in clinical trials or in use. Paramagnetic contrast agents serve to reduce T₁ and/or T₂. The capacity to differentiate regions or tissues that may be magnetically similar but histologically different is a major impetus for the preparation of these agents. These agents provide further contrast, and thus enhanced images, wherever the contrast agent is found. For example, the approved contrast agents outlined below may be injected into the circulatory system and used to visualize vascular structures and abnormalities, amongst other anatomical and physiological characteristics.

In the design of MRI agents, strict attention must be given to a variety of properties that will ultimately affect both the physiological fate of the agent and its ability to provide contrast enhancement. Two fundamental properties that must be considered are biocompatability and proton relaxation enhancement. Biocompatability is influenced by several factors including toxicity, stability (thermodynamic and kinetic), pharmacokinetics and biodistribution. Proton relaxation enhancement (or relaxivity) is chiefly governed by the choice of metal ion, the rotational correlation time of the contrast agent, the accessibility of the metal to surrounding water molecules and the time scale of the exchange of water between the metal ion and the bulk water population.

The measured relaxivity of the contrast agent is highly dependent on the selection of the metal ion. Paramagnetic metal ions, as a result of their unpaired electrons, act as potent relaxation enhancement agents. They decrease the T₁ relaxation times of nearby spins. The effect of the unpaired electrons falls off according to 1/r⁶, in which r is the radius between the electron and the water proton. Some paramagnetic ions decrease the T₁ without causing substantial line-broadening (e.g. gadolinium (III), (Gd³⁺)), while others induce significant line-broadening (e.g. superparamagnetic iron oxide). The mechanism of T₁ relaxation is generally a through space dipole-dipole interaction between the unpaired electrons of the paramagnet (the metal atom with an unpaired electron) and bulk water molecules (those water molecules not “bound” to the metal atom) that are in fast exchange with water molecules in the metal's inner coordination sphere (those water molecules bound to the metal atom).

The shortening of proton relaxation times by the Gd(III) ion is mediated by dipole-dipole interactions between its unpaired electrons and adjacent water protons. The effectiveness of the Gd(III) magnetic dipole drops off very rapidly as a function of its distance from these protons. Consequently, the protons which are relaxed most efficiently are those which are able to enter the first or second Gd(III) coordination spheres during the interval between the RF pulse and signal detection. For example, regions associated with a Gd(III) ion (nearby water molecules) appear bright in a MR image where the normal aqueous solution appears as dark background if the time between successive scans in the experiment is short (i.e., T₁ weighted image).

Localized T₂ shortening caused by superparamagnetic particles is believed to be due to the local magnetic field inhomogeneities associated with the large magnetic moments of these particles. Regions associated with a superparamagnetic iron oxide particle appear dark in an MR image where the normal aqueous solution appears as high intensity background if the echo time (TE) in the spin-echo pulse sequence experiment is long (i.e. T₂-weighted image).

The lanthanide ion Gd(III), has generally been chosen as the metal ion for contrast agents because it has a high magnetic moment (μ²=63BM²), a symmetric electronic ground state, (S⁸), and the largest paramagnetic dipole and the greatest paramagnetic relaxivity of any element. Gd(III) is chelated with an array of substances of diverse structure to render the complex substantially nontoxic. To date, a number of chelators have been used, including diethylenetriaminepentaacetic acid (DTPA), 1,4,7,10-tetraazacyclododecane′-N,N′N″,N′″-tetracetic acid (DOTA), and derivatives thereof. See, e.g., U.S. Pat. Nos. 5,155,215, 5,087,440, 5,219,553, 5,188,816, 4,885,363, 5,358,704, 5,262,532, and Meyer et al., Invest. Radiol. 25:S53 (1990). Although Gd(III) has generally been chosen as the metal atom for contrast agents, other metal ions present some advantages in certain situations. For example, Mn(II) displays greater relaxivity enhancement based on rotational correlation time and Dy(III) displays Curie Relaxation enhancement as fields where the relaxivity enhancement by Gd(III) is reduced.

The stability constant (K) for Gd(DTPA) is very high (logK=22.4) and is more commonly known as the formation constant (the higher the logK, the more stable the complex). This thermodynamic parameter indicates that the fraction of Gd(III) ions that are in the unbound state in a solution of Gd(DTPA) will be quite small. LogK should not be confused with the rate (kinetic stability) at which the loss of metal ion from the chelate occurs. The water soluble Gd(DTPA)-chelate is stable, substantially nontoxic, and one of the most widely used contrast enhancement agents in experimental and clinical imaging research. It was approved for clinical use in adult patients in June of 1988. It is an extracellular agent that accumulates in tissue by perfision-dominated processes. Image enhancement improvements using Gd(DTPA) are well documented in a number of applications (Runge et al., Magn, Reson. Imag. 3: 85 (1991); Russell et al., AJR 152: 813 (1989); Meyer et al., Invest. Radiol. 25: S53 (1990)), including visualizing blood-brain barrier disruptions caused by space occupying lesions and detection of abnormal vascularity. Gd(DTPA) was recently applied to the functional mapping of the human visual cortex by defining regional cerebral hemodynamics.

Another chelator used in Gd contrast agents is the macrocyclic ligand 1,4,7,10-tetraazacyclododecane-N,N′,N″N′″-tetracetic acid (DOTA). The Gd-DOTA complex has been thoroughly studied in laboratory tests involving animals and humans. The complex is conformationally rigid, has an extremely high formation constant (logK=28.5), and at physiological pH displays very slow dissociation kinetics.

As noted above, these MRI contrast agents have a variety of uses. Of particular interest are contrast agents that allow the visualization of a specific, selected component within a biological or other type of sample. Recently, contrast agents that are activatable by cellular or other processes have been disclosed. For example, Meade and co-workers have prepared and tested a prototypical biochemically-activated MRI contrast agent known as EGAD (U.S. Pat. No. 5,707,605). Biochemical activation of EGAD is reported to provide a modest increase in the relaxivity of the complex (approx. 20%). The time scale of the activation, however, is quite slow.

For diagnostic procedures, it is desirable to utilize biochemically-activated MRI agents that are activated on a time scale of minutes or shorter. Moreover, to minimize the necessary dosage of the agents, it is preferred to utilize components that undergo a marked alteration in relaxivity upon activation. Biochemically-activated MRI contrast agents are of interest to diagnose disease states and locate disease foci. Accordingly, it is generally desirable that the agent remains proximate to the site of activation for a time sufficient to allow its detection by MRI. Quite surprisingly, the present invention provides compounds having these desirable properties.

### BRIEF SUMMARY OF THE INVENTION

It has now been discovered that it is possible to rationally engineer properties of a biologically activated magnetic resonance contrast agent (BAMCA) such as the relaxivity increase or decrease on activation and the time scale on which activation occurs. Moreover, exemplary compounds of the invention remain at or near the region of interest in which they are activated.

The activation process is mediated by a biochemical species, e.g., an enzyme, an endogenous radical, or a process or a change in environment, e.g., a change in pH or pO₂. The compounds of the invention can be designed to respond to a selected biochemical activation pathway. For example, including an enzymatically or pH-sensitive bond within the compound structure provides compounds that respond to the presence of an enzyme or a change in pH. Moreover, the compounds can be designed to undergo a selective modulation in relaxivity within a predetermined time frame.

Exemplary BAMCA of the present invention are characterized by a change in relaxivity upon going from the unactivated to the activated state that is many fold higher than that of the art-recognized agents. Furthermore, in contrast to art-recognized agents, the relaxivity change upon activation can be either positive or negative. Moreover, exemplary compounds of the invention undergo activation at rates that are dramatically increased relative to art-recognized BAMCA.

For example, Meade et al. (U.S. Pat. No. 5,707,605) prepared a prototype BAMCA, EGAD (FIG. 4). The compound includes a Gd-DO3A signal-generating moiety that is linked to a glycosyl residue through a hydroxyethyl spacer. EGAD is reported to undergo an increase in relaxivity of about 20% upon activation, which occurs upon enzymatic cleavage of the glycosidic bond linking the hydroxyethyl spacer moiety to the glycosyl residue. The compound is very slowly activated and only in the presence of a large quantity of enzyme.

In contrast to EGAD, exemplary compounds of the invention undergo an increase in relaxivity of greater than about 40% upon activation. Moreover, the kinetics of activation for exemplary compounds of the invention are much faster than those of EGAD; the exemplary compounds undergo activation on a time scale of seconds to minutes.

Thus, in a first aspect, the invention provides an activatable BAMCA having the formula:
(MC-L)ₐ-X-(L¹-RM)_{b} (I)
in which MC is a metal chelate. The indices a and b represent integers that range from 1 to 4. The symbol RM represents a relaxivity-modulating group that includes at least one moiety that influences the relaxivity of the complex. The RM group influences complex relaxivity by modulating one or more complex characteristics, e.g., solubility, rate of tumbling (TR), hydrophobicity, hydrophilicity, affinity for interacting with endogenous species, e.g., proteins, membranes, and combinations of these mechanisms. In an exemplary embodiment, L is a hydrophobic moiety and RM is hydrophilic. When RM is cleaved from the complex, the effect of the hydrophobic character of L on complex relaxivity becomes more dominant than when the water-soluble RM was present. The increase in hydrophobicity of the complex often results in decreased complex water-solubility, which promotes increased complex aggregation and enhances the affinity of the complex for interacting with endogenous or exogenous biologically relevant entities, such as proteins, cells, membranes and the like. In addition to localizing the agent at the site of activation, the increased effective molecular size resulting from aggregation or interaction with a biologically relevant entity decreases the rate of tumbling of the complex, resulting in an increase in complex relaxivity. Even in the absence of changes in hydrophilicity or hydrophobicity changes in molecular weight can lead to significant relaxivity modulation.

In another exemplary embodiment, the MC includes a hydrophobic moiety and RM-L is a hydrophilic group that increases the overall hydrophilicity of the complex. When RM is cleaved from the remainder of the complex, the hydrophobicity (lipophilicity) of the complex increases. In an aqueous environment, the increased hydrophobicity enhances complex aggregation and interaction with hydrophobic biological structures, increasing the rotational correlation time and enhancing complex relaxivity. In an alternate complex, the RM is hydrophobic such that when it is cleaved the complex becomes more hydrophilic, tumbling is enhances and the rotational correlation time decreases, thereby lower the relaxivity of the complex.

Another mechanism that can be exploited to enhance complex relaxivity relies on the use of a relaxivity-modulating group that interacts with the paramagnetic metal ion to block one or more metal ion coordination site from interacting with water. When the relaxivity-modulating group is cleaved, the coordination site that it blocked becomes available to interact with water, leading to a complex with enhanced relaxivity. Alternatively, when the relaxivity-modulating group is occupying a coordination site of the metal ion, the relaxivity-modulating group can be drawn away from the metal coordination site by preferentially interacting with a target substance having specificity for the relaxivity-modulating moiety. The various mechanisms underlying changes in relaxation properties are not mutually exclusive, and a given compound of the present invention may operate using a combination of mechanisms, and may display synergism between two or more mechanisms.

The symbol L and L¹ represent independently selected linker arms. The symbol X represents a bond formed between the linker arm-metal chelate cassette (MC-L) and the linker arm-relaxivity modulating group cassette (L¹-RM). X is formed by the reaction of a reactive functional group on L and L¹. As will be appreciated by those of skill in the art, the linkage between MC and L, as well as that between L¹ and RM, is also formed by the reaction between a reactive functional group on each separate component.

When the relaxivity modulating group is removed from the remainder of the complex, the complex is converted into its activated form. The change in relaxivity (increase or decrease) upon activation of a complex according to Formula I is typically greater than about 15%, greater than about 20%, greater than about 25%, greater than about 30%, greater than about 35%, greater than about 40%, greater than about 45% or greater than about 50%.

In a second aspect, the invention provides an activated BAMCA having the formula:
MC-L-X¹ (II)
in which X¹ is a residue formed by the cleavage of cleaveable bond X-L¹. The other symbols are as described for Formula I. When activation enhances the relaxivity of a complex of the invention, an activated compound according to Formula II is greater than that of an unactivated compound according to Formula I by an amount that is typically greater than about 15%, greater than about 20%, greater than about 25%, greater than about 30%, greater than about 35%, greater than about 40%, greater than about 45% or greater than about 50%. These numbers are equally relevant in those embodiments in which the relaxivity of the complex decreases upon activation.

The invention also provides “precursors” to the compounds of Formula I in which the chelating moiety is unmetalated.

Also provided is a method of performing a MR imaging experiment on a subject, tissue, cell or other sample of biological or other origin. The method includes administering to the subject, tissue, cell or sample a compound according to Formula I and acquiring an MRI data set.

Other aspects, embodiments and advantages of the present invention will be apparent from the detailed description that follows.

### BRIEF DESCRIPTION OF THE DRAWINGS

FIG. 1 is an overview of the factors that can be modulated in exemplary biochemically-activated MRI contrast agents of the invention.

FIG. 2 is a scheme showing the transition from low q to high q states of a DOTA-based biochemically-activated MRI contrast agent.

FIG. 3 is a scheme showing the transition from a high rotational correlation times state to a low rotational correlation time state of DTPA- and DOTA-based biochemically-activated MRI contrast agents.

FIG. 4 is a comparison of the properties of an art-recognized biochemically-activated MRI contrast agent and a biochemically-activated MRI contrast agent of the invention, compound I.

FIG. 5 is a synthetic scheme for the preparation of compound I.

FIG. 6 is a LC-MS trace for compound I, showing the purity of the complex.

FIG. 7 is a LC-MS trace for compound I, showing the stability of the complex to storage.

FIG. 8 is a plot of the T₁ and T₂ relaxivities of compound I in pH 7.4 HEPES and 4.5% HSA. The table compares the T₁ and T₂ relaxivities of compound I in pH 7.4 HEPES and in pH 7.4 HEPES and 4.5% HSA.

FIG. 9 is plot of the change in relaxivity vs. time for an exemplary compound of the invention.

FIG. 10 is a table of representative compounds of the invention placed in the context of their activation time scale. Also displayed for comparison is the art-recognized compound EGAD.

FIG. 11 is the structure of an exemplary galactosidase-activated BAMCA of the invention.

FIG. 12 is a plot showing the relaxivity modulation of the BAMCA of FIG. 11 by β-galactosidase (no albumin).

FIG. 13 is a plot showing the relaxivity modulation of the BAMCA of FIG. 11 by β-galactosidase (with albumin).

FIG. 14 is the structure of an exemplary protease-activated BAMCA of the invention.

FIGS. 15A and 15B are plots showing the relaxivity modulation of the BAMCA of FIG. 14 by the protease MMP-7.

FIG. 16 is the structure of an exemplary protease-activated dimeric BAMCA of the invention.

FIG. 17 is a plot showing the relaxivity modulation of the BAMCA of FIG. 16 by the protease MMP-7.

### DETAILED DESCRIPTION OF THE INVENTION

Definitions

“Activate” and “activation,” as used herein, refer to a process that results in a change of complex relaxivity. The relaxivity can increase or decrease in response to the activation. Exemplary mechanisms of activation include cleaving a water-soluble moiety from the complex (e.g., cleaving a hydrophilic group from a hydrophobic linker attached to the metal chelate); cleaving a hydrophobic group from the complex (e.g., cleaving a hydrophobic group from a hydrophilic linker); and freeing at least one coordination site of a paramagnetic metal ion of a compound of the invention from its interaction with the relaxivity-modulating group.

The term “relaxivity-modulating group,” refers to a group, which, in an unactivated BAMCA either increases or decreases the relaxivity of the unactivated BAMCA relative to the relaxivity of the corresponding activated BACMA. When the complex is activated, the relaxivity-modulating group, or the bond that attaches it to the remainder of the complex, interacts with a target substance in a subject, tissue, cell or other sample to alter the complex relaxivity relative to the relaxivity of the compound in its unactivated state. The RM can be cleaved by this interaction or it can be reversibly or irreversibly bound to the target substance. Exemplary relaxivity-modulating groups enhance the water-solubility of the complex, or prevent the interaction between bulk water and a coordination site of a paramagnetic complex of the invention. Further exemplary relaxivity-modulating groups are biochemically relevant species, e.g., peptides, amino acids, nucleic acids, saccharides, and the like.

The term “stable, chelated form” refers to metal ion that is bound to an organic chelating agent. The complex is essentially undissociated into its constituent metal ion and chelating agent over the time course of a selected MR imaging experiment. It is generally preferred that no more than 10%, preferably no more than 5% and more preferably no more than 1% of a dose of a BAMCA administered to a subject in which the metal ion is in a “stable, chelated form” dissociates from the chelating agent over the time course of the MR imaging experiment.

“Peptide” refers to a polymer in which the monomers are amino acids and are joined together through amide bonds, alternatively referred to as a polypeptide. Additionally, unnatural amino acids, for example, β-alanine, phenylglycine and homoarginine are also included. Amino acids that are not gene-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer. The L-isomer is generally preferred. In addition, other peptidomimetics are also useful in the present invention. As used herein, “peptide” refers to both glycosylated and unglycosylated peptides. Also included are peptides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

As used herein, “nucleic acid” means any natural or non-natural nucleoside, or nucleotide and oligomers and polymers thereof, e.g., DNA, RNA, single-stranded, double-stranded, triple-stranded or more highly aggregated hybridization motifs, and any chemical modifications thereof. Modifications include, but are not limited to, conjugation with a compound of the invention or a construct that includes a compound of the invention covalently attached to a linker that tethers the compound to the nucleic acid, and those providing the nucleic acid with a group that incorporates additional charge, polarizability, hydrogen bonding, electrostatic interaction, fluxionality or functionality to the nucleic acid. Exemplary modifications include the attachment to the nucleic acid, at any position, of one or more hydrophobic or hydrophilic moieties, minor groove binders, intercalating agents, quenchers, chelating agents, metal chelates, solid supports, and other groups that are usefully attached to nucleic acids.

Where chemical moieties are specified by their conventional chemical formulae, written from left to right, they equally encompass the moiety which would result from writing the structure from right to left, e.g., —CH₂O— is intended to also recite —OCH₂—; —NHS(O)₂— is also intended to represent. —S(O)₂HN—, etc.

The term “alkyl,” by itself or as part of another substituent, means, unless otherwise stated, a straight or branched chain, or cyclic hydrocarbon radical, or combination thereof, which may be fully saturated, mono- or polyunsaturated and can include mono-, di- and multivalent radicals, having the number of carbon atoms designated (i.e. C₁-C₁₀ means one to ten carbons). Examples of saturated alkyl radicals include, but are not limited to, groups such as methyl, methylene, ethyl, ethylene, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, includes “alkylene” and those derivatives of alkyl defined in more detail below, such as “heteroalkyl.” Alkyl groups, which are limited to hydrocarbon groups, are termed “homoalkyl”.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R^{o}C(O)₂—.

The terms “cycloalkyl” and “heterocycloalkyl”, by themselves or in combination with other terms, represent, unless otherwise stated, cyclic versions of “alkyl” and “heteroalkyl”, respectively. Also included are di- and multi-valent species such as “cycloalkylene.” Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, species such as trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Also included are di- and multi-valent linker species, such as “arylene.” Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) (“alkyl group substituents”) can be one or more of a variety of groups selected from, but not limited to: —OR′, ═O, ═NR′, ═N—OR′, —NR′R″, —SR′, -halogen, —SiR′R″ R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″ R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups (“aryl group substituents”) are varied and are selected from, for example: halogen, —OR′, ═O, ═NR′, ═N—OR′, —NR′R″, —SR′, -halogen, —SiR′R″ R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)=NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

“Target substance,” as utilized herein refers to the species of interest in a MRI experiment. The term refers to a substance, which is detected qualitatively or quantitatively using a compound, complex or method of the present invention. Examples of such substances include cells and portions thereof, enzymes, antibodies, antibody fragments and other biomolecules, e.g., antigens, polypeptides, glycoproteins, polysaccharides, complex glycolipids, nucleic acids, effector molecules, signaling and receptor molecules, ionic species, radicals, enzymes inhibitors and the like, and drugs, pesticides, herbicides, agents of war and other bioactive agents.

As used herein, “pharmaceutically acceptable carrier” includes any material, which when combined with the conjugate retains the conjugates' activity, is well tolerated, and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, intranasal or subcutaneous administration, or the implantation of a slow-release device e.g., a mini-osmotic pump, to the subject. Administration is by any route including parenteral, and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, intrathecal, intraarticular, and intracranial. Moreover, where injection is to characterize a tumor, administration may be directly to the tumor and/or into tissues surrounding the tumor. Other modes of delivery include, but are not limited to, the use of oil-in-water lipid emulsions, liposomal formulations, intravenous infusion, transdermal patches, etc.

### The Embodiments

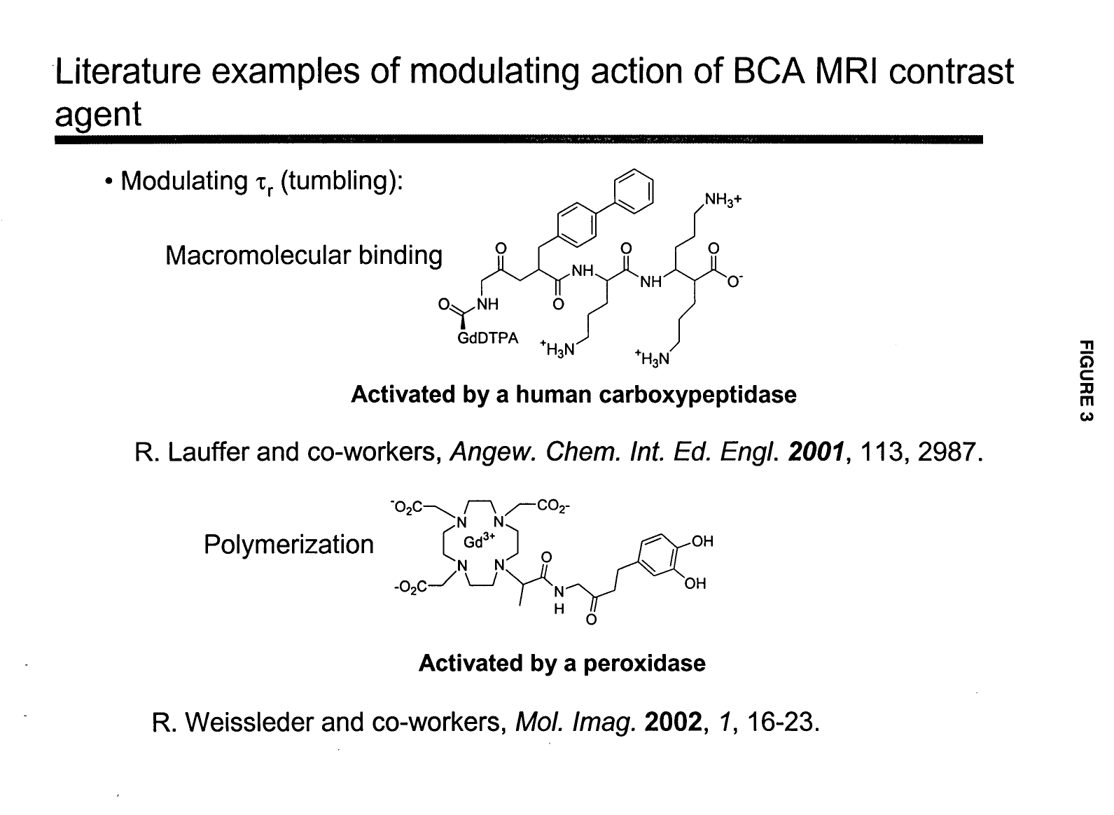

The present invention provides magnetic resonance imaging contrast agents that detect target substances, e.g., biochemical species. The agents, referred to herein as biochemically-activated MRI contrast agents (BAMCA), are activated in the presence of a target substance, e.g., an enzyme. The activation changes the relaxivity of the agent, thereby altering the MR image in the presence of the target substance (FIG. 3).

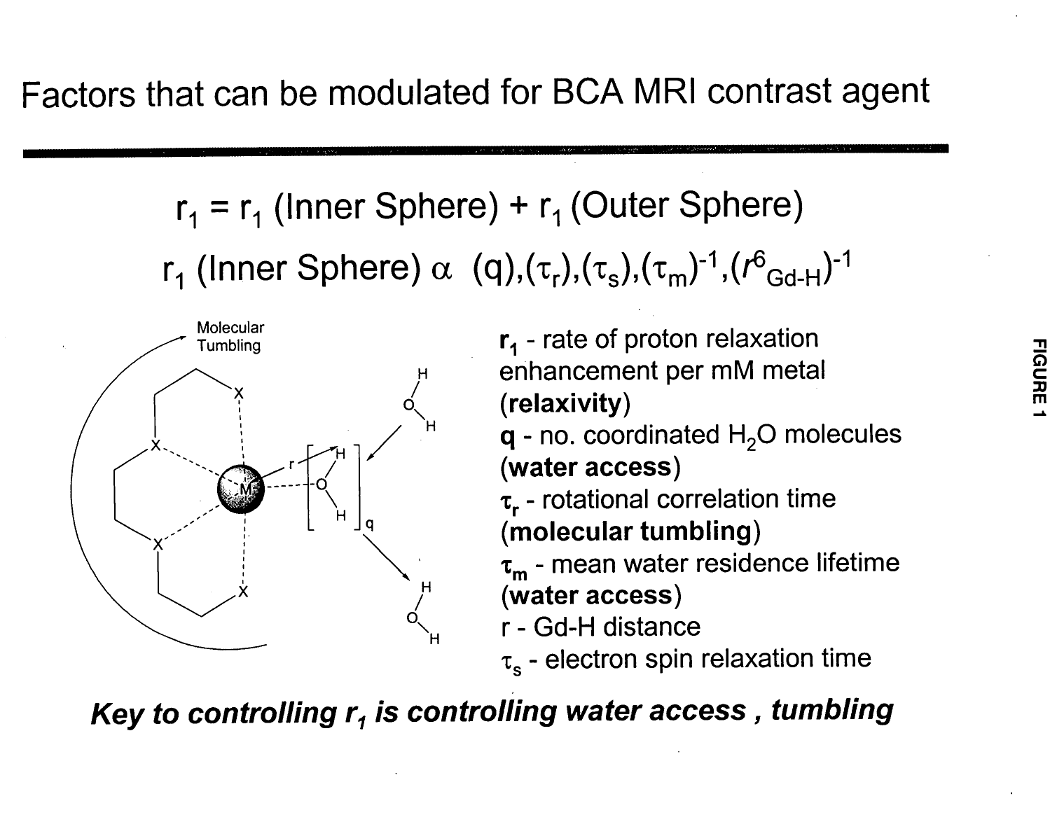

The activation of the contrast agent in the presence of the target substance is based on a change in one or more properties of the contrast agent occurring as a result of the agent's interaction with the target substance. Exemplary contrast agent properties that are altered as a result of activation include the rotational correlation time, and the dynamic equilibrium that affects the rate of exchange of water molecules in one or more coordination sites of a paramagnetic metal ion complex of the invention (FIG. 1). Activation may also change the physical properties of the contrast agent, resulting in changes in solubility, binding to endogenous components such as cell membranes or plasma proteins, and changes in biolocalization and clearance.

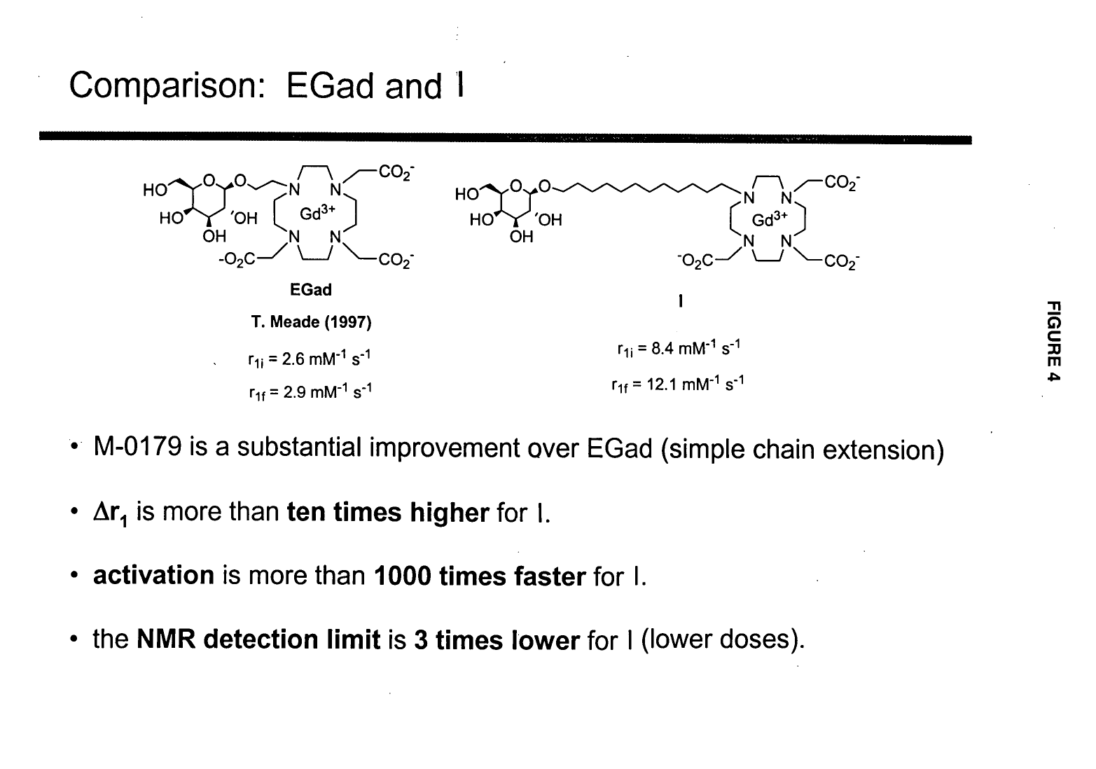

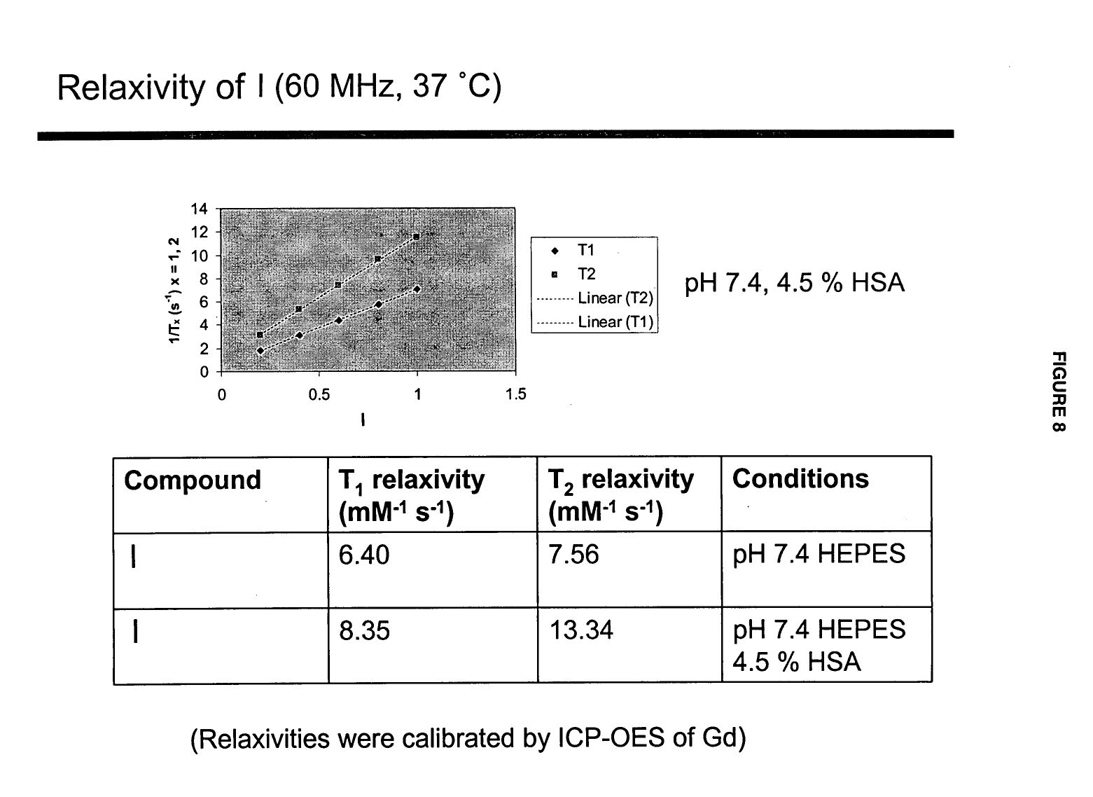

In some embodiments of the present invention, the rate of complex tumbling or water exchange is influenced by the presence or absence of the target substance in the surrounding environment. An exemplary complex is functionalized with a hydrophilic relaxivity-modulating group that is bound to the metal chelate through a hydrophobic linker. In a selected embodiment, a complex according to this design is activated by the target substance, which cleaves the relaxivity-modulating group from a lipophilic linker, thereby activating (increasing the lipophilicity) of the complex. See, for example, compound I of FIG. 4. The relaxivity of the complex of FIG. 4 increases upon cleavage of the relaxavity-modulating group from the BAMCA (FIG. 8).

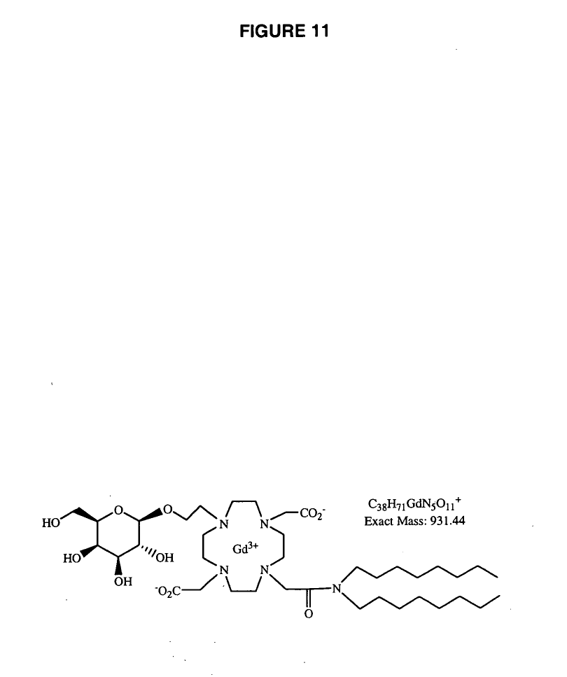

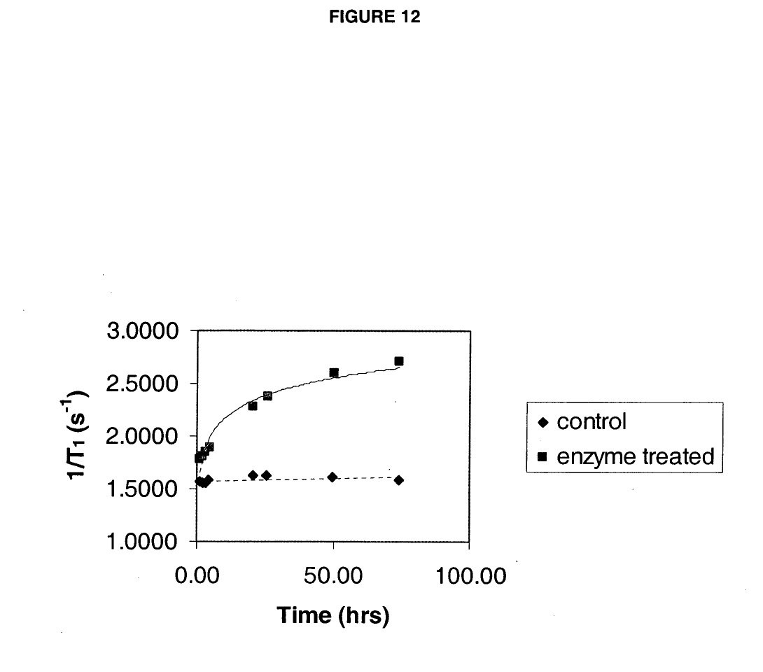

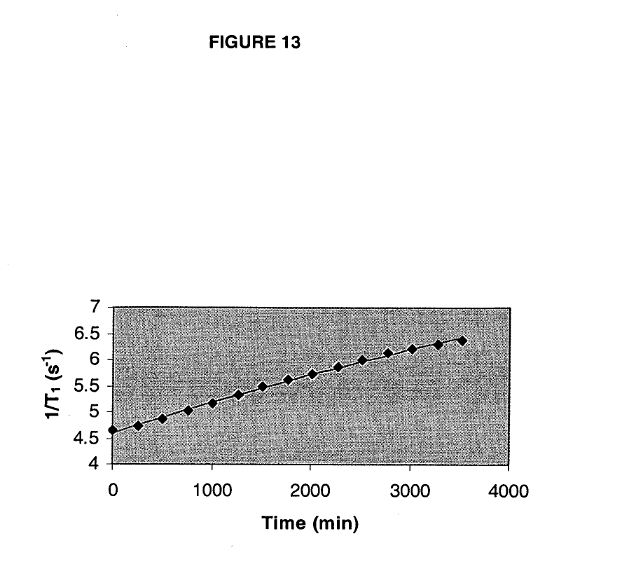

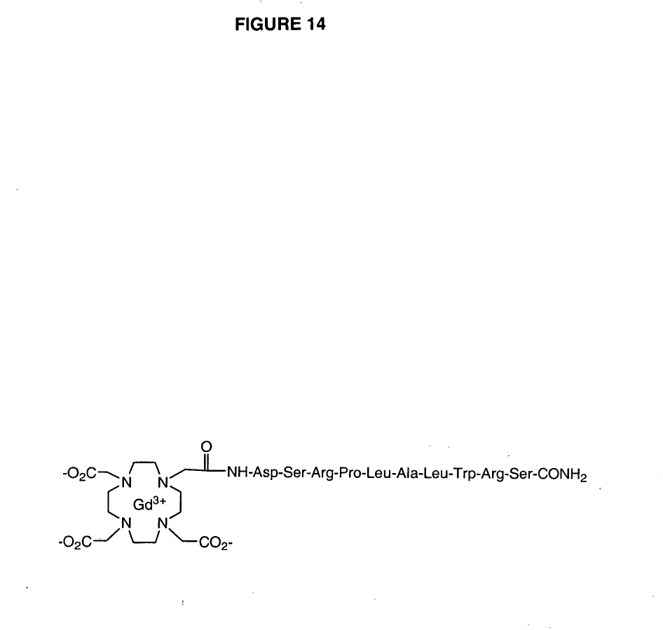

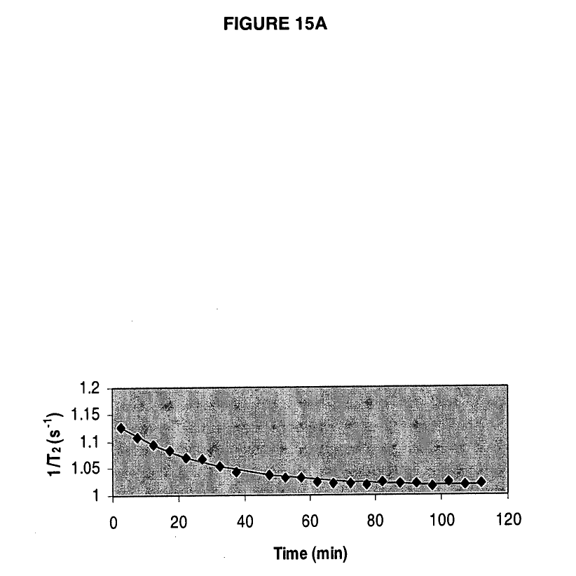

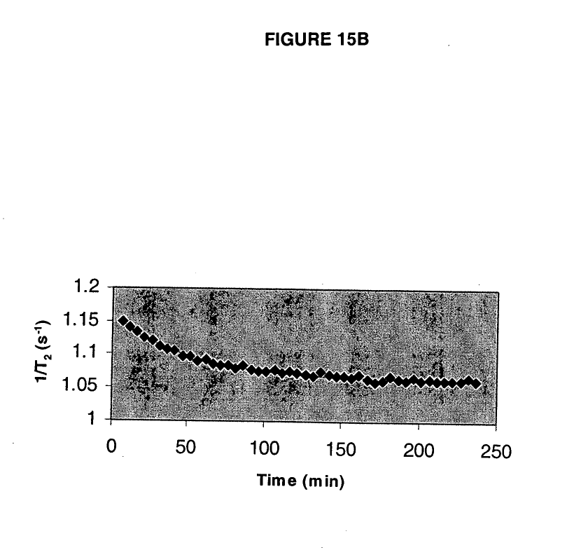

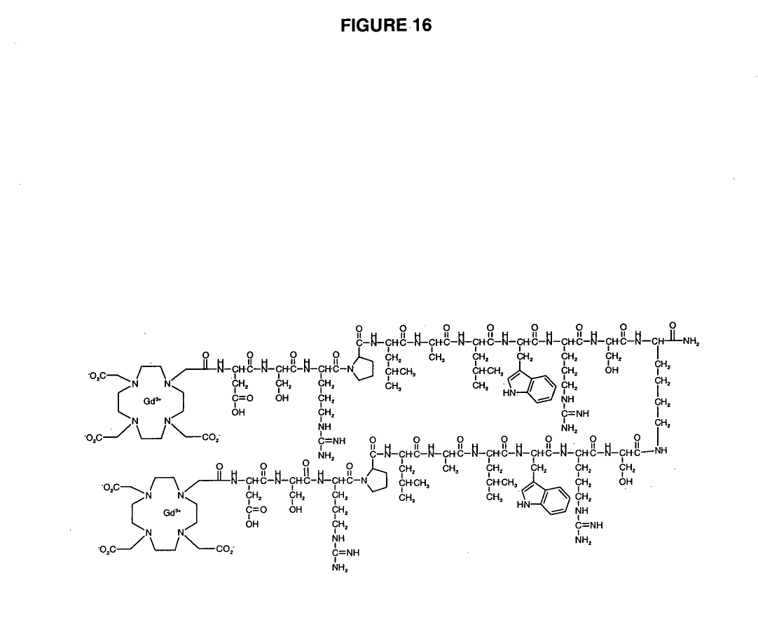

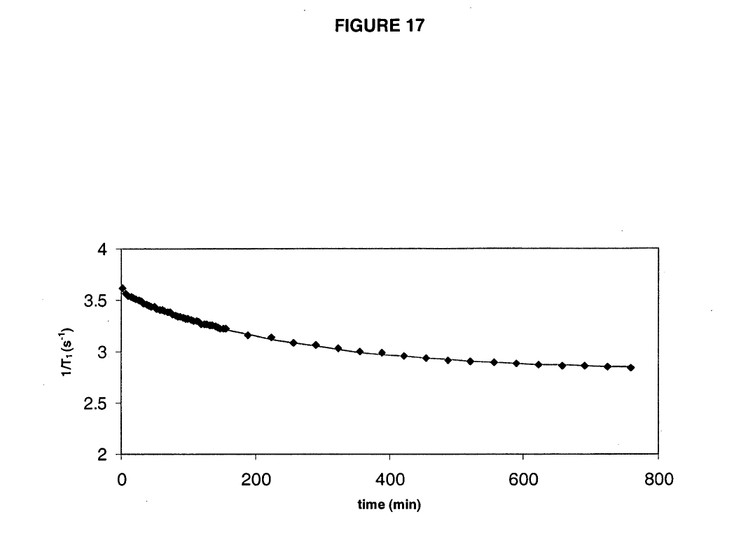

In another exemplary embodiment, the BAMCA of the invention includes a hydrophobic region and a hydrophilic region. Cleavage of either the hydrophobic region or hydrophilic region from the metal chelate results in modulation of the BAMCA relaxivity-thus in this embodiment, the cleaved group is operationally defined as the relaxivity-modulating group. See, for example, FIG. 11.

Exemplary hydrophilic and hydrophobic moieties that are of use as of the BACMA of the invention include substituted or unsubstituted alkyl and substituted or unsubstituted heteroalkyl residues. Exemplary substituted or unsubstituted alkyl moieties include C₆-C₃₀ residues. Exemplary substituted or unsubstituted heteroalkyl moieties include polyethers, e.g., poly(ethylene glycol), methoxy-poly(ethylene glycol) and the like.

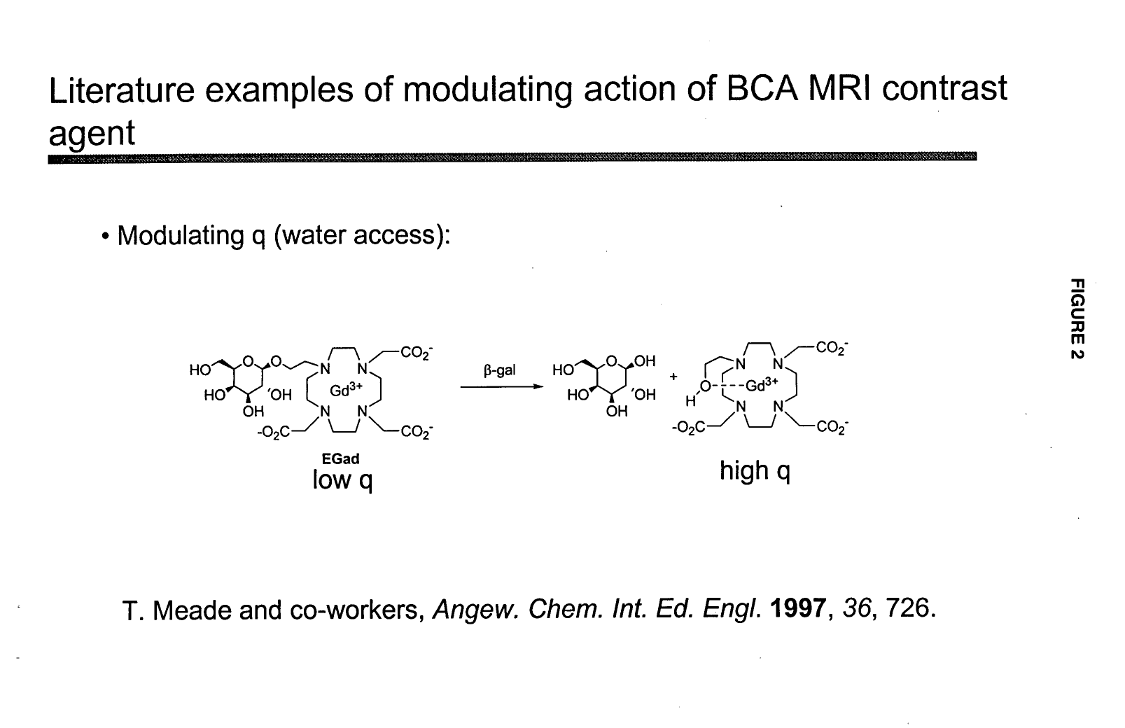

In another embodiment, the metal ion in the unactivated paramagnetic metal ion complex of the invention has fewer coordination sites available for interaction with water molecules of the local environment because a coordination site of the unactivated chelate is occupied or blocked by the coordinating atoms of the chelator and at least one relaxivity-modulating group. In this embodiment of the invention, upon activation of a BAMCA, the target substance sequesters or cleaves the relaxivity-modulating group, freeing a coordination site on the metal ion (FIG. 2). The concomitant increase in the population of water molecules in the metal ion inner coordination sphere causes an increase in the relaxivity of the metal ion, thereby producing image enhancement which corresponds to the presence of the target substance.

Generally, a 2 to 5% change in the MRI signal used to generate the image is enough to be detectable. Thus, it is preferred that the agents of the invention, in the presence of a target substance, modify the MRI signal by at least about 2 to about 5% as compared to the signal in the absence of the target substance. Signal change of 2 to 15% is preferred, 10 to 20% is more preferred, 25 to 35% still more preferred, and 40-50% is even more preferred for each activatable agent.

The time-scale of activation of the compounds of the present invention is also relevant. For example, known BAMCA are activated slowly, a feature that can compromise their utility in certain diagnostic procedures. Exemplary BAMCA of the present invention are activated on a time-scale of seconds to minutes. Thus, in an exemplary embodiment, the invention provides a BAMCA at least 30%, preferably at least 40% and more preferably at least 50% of an administered dose of which is activated within 20 minutes following contact with the target species.

The complexes of the invention comprise a chelator and a relaxivity-modulating group joined by a linker arm that optionally includes a cleaveable bond. Thus, in a first aspect, the invention provides an inactivated BAMCA having the formula:
(MC-L)ₐ-X-(L¹-RM)_{b} (I)
in which MC is a metal chelate. The indices a and b represent integers that range from 1 to 4. The symbol RM represents a relaxivity-modulating group that includes at least one moiety that influences the relaxivity of the complex. The RM group influences complex relaxivity by modulating one or more complex characteristics, e.g., solubility, rate of tumbling (τ_{R}), affinity for interacting with endogenous species, e.g., proteins, membranes, and combinations of these mechanisms. In an exemplary embodiment, L is a hydrophobic moiety and RM is hydrophilic. When RM is cleaved from the complex, the effect of the hydrophobic character of L on complex relaxivity becomes more dominant than when the water-soluble RM was present. The increase in hydrophobicity of the complex often results in decreased complex water-solubility, which promotes increased complex aggregation and enhances the affinity of the complex for interacting with endogenous or exogenous biologically relevant entities, such as proteins, cells, membranes and the like. In addition to localizing the agent at the site of activation, the increased effective molecular size resulting from aggregation or interaction with a biologically relevant entity decreases the rate of tumbling of the complex, resulting in an increase in complex relaxivity. Even in the absence of changes in hydrophilicity or hydrophobicity changes in molecular weight can lead to significant relaxivity modulation

Another mechanism that can be exploited to enhance complex relaxivity relies on the use of a relaxivity-modulating group that interacts with the paramagnetic metal ion to block one or more metal ion coordination site from interacting with water. When the relaxivity-modulating group is cleaved, the coordination site that it blocked becomes available to interact with water, leading to a complex with enhanced relaxivity. Alternatively, when the relaxivity-modulating group is occupying a coordination site of the metal ion, the relaxivity-modulating group can be drawn away from the metal coordination site by preferentially interacting with a target substance having specificity for the relaxivity-modulating moiety. The various mechanisms underlying changes in relaxation properties are not mutually exclusive, and a given compound of the present invention may operate using a combination of mechanisms, and may display synergism between two or more mechanisms.

The symbol L and L¹ represent independently selected linker arms. The symbol X represents a bond formed between the linker arm-metal chelate cassette (MC-L) and the linker arm-relaxivity modulating group cassette (L¹-RM). X is formed by the reaction of a reactive functional group on L and L¹. As will be appreciated by those of skill in the art, the linkage between MC and L, as well as that between L¹ and RM, is also formed by the reaction between a reactive functional group on each separate component. In selected embodiments, it is generally preferred that the linker arm is of a length sufficient to prevent the RM from interacting with an open coordination site on the paragmagnetic metal ion, which leads to any substantial decrease in water population at the metal center. Exemplary linker arms will be at least 3, preferably at least 4 and even more preferably at least 5 atoms atoms in length. In this embodiment, a preferred linker is one that prevents a metal complexing moiety of the RM (e.g., O, N, S) from complexing with the metal center through the formation of a five- or six-membered ring.

In an exemplary BAMCA, the relaxivity-modulating group is joined to the linker arm through X, which is a cleaveable bond. The cleavable bond may be attached directly to the relaxivity-modulating group in a zero-order fashion or it may form a component of the linker arm, such that cleavage results in the scission of the linker arm. Many cleavable groups are known in the art. See, for example, Jung et al., Biochem. Biophys. Acta 761: 152-162 (1983); Joshi et al., J. Biol. Chem. 265: 14518-14525 (1990); Zarling et al., J. Immunol. 124: 913-920 (1980); Bouizar et al., Eur. J. Biochem. 155: 141-147 (1986); Park et al., J. Biol. Chem. 261: 205-210 (1986); Browning et al., J. Immunol. 143: 1859-1867 (1989). Moreover a broad range of cleavable, bifunctional (both homo- and hetero-bifunctional) linker groups is commercially available from suppliers such as Pierce.

Exemplary cleaveable moieties can be cleaved using light, heat, pH changes, enzymes or reagents such as thiols, hydroxylamine, bases, periodate and the like. Moreover, certain preferred groups are cleaved in vivo in response to being endocytized (e.g., cis-aconityl; see, Shen et al., Biochem. Biophys. Res. Commun. 102: 1048 (1991)). Exemplary cleaveable moieties include disulfides, esters, imides, amides, carbonates, and glycosides. The metal ion complexes of the invention comprise a paramagnetic metal ion bound to a complex comprising a chelator and a relaxivity-modulating group. By “paramagnetic metal ion”, “paramagnetic ion” or “metal ion” herein is meant a metal ion magnetized parallel or antiparallel to a magnetic field to an extent proportional to the field. Generally, these are metal ions that have unpaired electrons; this is a term understood in the art. Examples of suitable paramagnetic metal ions, include, but are not limited to, gadolinium (Gd⁺³), iron (Fe⁺³), manganese (Mn⁺²), yttrium (Y⁺³), dysprosium (Dy⁺³), and chromium (Cr⁺³). In an exemplary embodiment the paramagnetic ion is Gd⁺³, due to its high magnetic moment, a symmetric electronic ground state, and its current approval for diagnostic use in humans. Manganese (Mn⁺²) possesses advantages when molecular tumbling is modulated and dysprosium (Dy⁺³) displays enhanced relaxivity at high magnetic fields.

The relaxivity-modulating group influences complex relaxivity by modulating one or more complex characteristics, e.g., solubility, rate of tumbling (TR), affinity for interacting with endogenous species, e.g., proteins, membranes, number of waters coordinated to the metal ion, and combinations of these mechanisms. In an exemplary embodiment, L is a hydrophobic moiety and RM is hydrophilic. When RM is cleaved, the effect of the hydrophobic character of L on complex relaxivity is more dominant than when the water-soluble RM was present. The increase in hydrophobicity of the complex can result in decreased complex water-solubility, which promotes increased complex aggregation and enhances the affinity of the complex for interacting with endogenous or exogenous biologically relevant entities, such as proteins, cells, membranes and the like. The increased effective molecular size resulting from aggregation or interaction with a biologically relevant entity decreases the rate of tumbling of the complex, resulting in an increase in complex relaxivity.

Exemplary relaxivity-modulating groups are also capable of interacting with a target substance. The relaxivity-modulating group has an affinity for the target substance, such that the water solubility of the group is masked, or the group ceases blocking or occupying at least one coordination site of the metal ion complex when the target substance is present. In the presence of the target substance, the relaxivity-modulating group associates or interacts with the target substance and is released from its association with the metal ion, thus freeing at least one coordination site of the metal ion such that the rapid exchange of water can occur at this site, resulting in image enhancement.

A typical relaxivity-modulating group has a bond that interacts with a target substance, e.g., the substructure RM-L¹-X—, or a component of this substructure, is a substrate for an enzyme, an antigen for an antibody, a ligand for a receptor, etc. The bond may or may not play a role in chelation of the paramagnetic metal ion.

The nature of the interaction between the relaxivity-modulating group and the target substance depends on the target substance to be detected or visualized via MRI. For example, suitable target substances include, but are not limited to, enzymes; proteins; peptides; ions such as Ca⁺², Mg⁺², Zn+, Cl⁻, and Na⁺; cAMP; radicals such as NO, peroxynitrite, and superoxide; receptors such as cell-surface receptors and ligands; hormones; antigens; antibodies; ATP; NADH; NADPH; FADH₂; FNNH₂; coenzyme A (acyl CoA and acetyl CoA); and biotin, among others. The complexes of the present invention and the use of these complexes in the methods disclosed herein are not limited by the nature of the target substance. Those of skill in the art can readily combine their knowledge of substances that selectively interact with a target substance and the compound motifs, and mechanisms of action, set forth herein to design additional BACMA that fall within the ambit of the present invention.

In some embodiments, the interaction between the relaxivity-modulating group and the target substance is irreversible, such that the relaxivity-modulating group does not reassociate with the complex; for example, when the group includes, or is attached to remainder of the complex through, an enzyme substrate that is cleaved upon exposure to a target enzyme. Alternatively, the interaction is reversible, such that the relaxivity-modulating group operates without a permanent change in structure, for example, when the relaxivity-modulating group comprises a ligand that can bind reversibly to the paramagnetic metal ion

In an exemplary embodiment, the target substance is an enzyme, and the relaxivity-modulating group, or a bond linking it to another component of the complex, is an enzyme substrate. In this embodiment, the relaxivity-modulating group is separated from the metal ion complex of the invention by the action of the enzyme. When the relaxivity-modulating group is hydrophilic, the separation enhances the hydrophobicity of the complex. Alternatively, the action of the enzyme exposes a coordination site on the metal ion that was blocked by interaction with the relaxavity-modulating group. This embodiment allows the amplification of the image modulation attributable to the target substance since a single molecule of the target substance is able to generate many activated metal ion complexes.

As will be appreciated by those skilled in the art, numerous enzyme target substances can be detected using a compound of the invention. The target substance enzyme may be chosen on the basis of a correlation to a disease condition, for example, for diagnostic purposes. Alternatively, the metal ion complexes of the present invention may be used to establish such correlations. Suitable classes of enzymes include, but are not limited to, hydrolases such as proteases, carbohydrases, nucleases, esterases, lipases; isomerases such as racemases, epimerases, tautomerases, or mutases; transferases, kinases and phophatases.

Exemplary enzymes associated with disease states that are detectable by a compound of the invention include enzymes associated with the generation or maintenance of arterioschlerotic plaques and lesions within the circulatory system, inflammation, wounds, immune response, and tumors. Enzymes such as lactase, maltase, sucrase or invertase, α-amylase, aldolases, β-glucuronidase, β-galactosidase, glycogen phosphorylase, kinases such as hexokinase, proteases such as serine, cysteine, aspartyl and metalloproteases may also be detected, including, but not limited to, matrilysin, trypsin, chymotrypsin, and other therapeutically relevant serine proteases such as tPA and the other proteases of the thrombolytic cascade; the cathepsins, including cathepsin B, L, S, H, J, N and O; and calpain. Similarly, bacterial and viral infections may be detected via characteristic bacterial and viral enzymes.

Once the target enzyme is identified or selected, well-known parameters of enzyme substrate specificities can be used to design relaxivity-modulating groups and to choose the bonds to attach these groups another component of the compound. For example, when the enzyme target substance is a protease, the relaxivity-modulating group may be a peptide or polypeptide which is capable of being cleaved by the target protease or, alternatively, the relaxivity-modulating group can be bound to another component of the compound through a peptide that is a protease substrate. In an exemplary embodiment, “X” is a peptide or peptide-mimetic bond that is cleaved by the protease. Exemplary embodiments utilize peptides that include about 2 to about 15 amino acid residues, about 2 to about 8 amino acid residues, or about 2 to about 4 amino acid residues.

When the enzyme target substance is a carbohydrase (e.g., glycosidase), an exemplary relaxivity-modulating group is a carbohydrate group capable of being cleaved by the target carbohydrase. Alternatively, the relaxivity-modulating group is attached to another component of the compound through a carbohydrate, a glycosidic linkage or a carbohydrate- or glycosidic linkage-mimetic. For example, when the enzyme target is lactase or β-galactosidase, an exemplary enzyme substrate relaxivity-modulating group is lactose or galactose. Similar enzyme/substrate pairs include sucrase/sucrose, maltase/maltose, and α-amylase/amylose.

In another exemplary embodiment, the relaxivity-modulating group is an enzyme inhibitor, such that, in the presence of the enzyme, the inhibitor relaxivity-modulating group disassociates from the metal ion complex to interact or bind to the enzyme, thus freeing an inner coordination sphere site of the metal ion for interaction with water. Alternatively, the interaction of the inhibitor with the enzyme increases the effective molecular size of the complex. Similar to the compounds discussed above, the enzyme inhibitors in this embodiment are chosen on the basis of the enzyme target substance and the corresponding known characteristics of the enzyme.

In a further exemplary embodiment, the target substance is a physiologically-relevant agent other than an enzyme. For example, in the enzyme/substrate embodiment, the physiological agent interacts with the relaxivity-modulating group of the BAMCA, such that in the presence of the physiological agent, there is rapid exchange of water in at least one inner sphere coordination site of the metal ion complex, or a change in the rotational correlation time of the complex. Thus, for example, the target substance may be a physiologically active ion, and the relaxivity-modulating group is an ion binding ligand. For example, the target substance may be the Ca⁺² ion, and the relaxivity-modulating group may be a calcium binding ligand such as is known in the art (see Grynkiewicz et al., J. Biol. Chem. 260(6): 3440-3450 (1985); Haugland, R. P., Molecular Probes Handbook of Fluorescent Probes and Research Chemicals (1989-1991)). Other suitable target ions include Mn⁺², Mg⁺², Zn⁺², Na⁺, and Cl⁻.

When Ca⁺² is the target substance, preferred relaxivity-modulating groups include, but are not limited to, bis(o-amino-phenoxy)ethane-N,N,N′,N′-tetraacetic acid (BAPTA), ethylene glycol bis(P-aminoethyl ether)-N,N,N′,N′-tetraacetic acid (EGTA); ethylenediaminetetracetic acid (EDTA); and derivatives thereof, such as disclosed in Tsien, Biochem. 19: 2396-2404 (1980).

Potential relaxivity-modulating groups are easily tested to see if they are functional in the compounds and methods of the invention. Thus, for example, complexes (or chelates) are made with potential relaxivity-modulating moieties and then compared with an analogous complex (or chelate) without the relaxivity-modulating group in relaxometry or imaging experiments. Once it is shown that the relaxivity-modulating group can be altered or removed to effect activation, the target substance is added and the experiments repeated to show that interaction with the target substance changes the relaxivity properties and thus enhances the image.

The linkers, L and L¹, can be of any useful length, including zero order. Exemplary linker arms include substituted and unsubstituted alkyl, substituted and unsubstituted heteroalkyl, substituted and unsubstituted aryl or substituted and unsubstituted heteroaryl groups. The linker arms can be attached to the other components of the compounds of the invention at any point either internal to a chain structure or at a terminus of the linker chain. In a further exemplary embodiment, the linker arm is a substituted and unsubstituted alkyl or substituted and unsubstituted heteroalkyl moiety that includes about 3 to about 30, preferably about 6 to about 20 and more preferably from about 8 to about 16 atoms.

The choice of metal chelate (MC) is determined by convenience and the nature of the application in which the compounds of the invention are to be used and, generally, is not critical to the invention. Many useful chelating groups, crown ethers, cryptands and the like are known in the art and can be incorporated into the compounds of the invention (e.g., EDTA, DTPA, DOTA, NTA, HDTA, etc. and their phosphonate analogs such as DTPP, EDTP, HDTP, NTP, etc). See, for example, Pitt et al., “The Design of Chelating Agents for the Treatment of Iron Overload,” In, INORGANIC CHEMISTRY IN BIOLOGY AND MEDICINE; Martell, Ed.; American Chemical Society, Washington, D.C., 1980, pp. 279-312; Lindoy, THE CHEMISTRY OF MACROCYCLIC LIGAND COMPLEXES; Cambridge University Press, Cambridge, 1989; Dugas, BIOORGANIC CHEMISTRY; Springer-Verlag, New York, 1989; Alexander, Chem. Rev. 95: 273-342 (1995); Jackels, Pharm. Med. Imag, Section III, Chap. 20, p. 645 (1990); Meyer et al., Invest. Radiol. 25: S53 (1990); U.S. Pat. Nos. 5,155,215, 5,087,440, 5,219,553, 5,188,816, 4,885,363, 5,358,704, 5,262,532, and references contained therein.

When the metal ion is Gd⁺³, a preferred chelator is based upon the 1,4,7,10-tetraazacyclododecane structure, such as the N,N′,N″,N′″-tetracetic acid (DOTA) or N,N′,N″-triacetic acid (DO3A). Other suitable Gd⁺³ chelators are described in Alexander, supra, Jackels, supra, U.S. Pat. Nos. 5,155,215, 5,087,440, 5,219,553, 5,188,816, 4,885,363, 5,358,704, 5,262,532, and Meyer et al., Invest. Radiol. 25:S53 (1990), among others.

When the paramagnetic ion is Fe⁺³, appropriate chelators will have 6 or fewer coordination atoms, since Fe⁺³ is capable of binding 6 coordination atoms. Suitable chelators for Fe⁺³ ions are well known in the art, see for example Lauffer et al., J. Am. Chem. Soc. 109: 1622 (1987); Lauffer, Chem. Rev. 87: 901-927 (1987); and U.S. Pat. Nos. 4,885,363, 5,358,704, and 5,262,532, all which describe chelators suitable for Fe(III).

When the paramagnetic ion is Mn⁺², appropriate chelators will have 6 or fewer coordination atoms, since Mn⁺² is capable of binding 6 or 7 coordination atoms. Suitable chelators for Mn⁺² ions are well known in the art; see for example, Lauffer, Chem. Rev. 87: 901-927 (1987) and U.S. Pat. Nos. 4,885,363, 5,358,704, and 5,262,532.

When the paramagnetic ion is Y⁺³, appropriate chelators will have 8 or fewer coordination atoms, since Y⁺³ is capable of binding 8 or 9 coordination atoms. Suitable chelators for Y⁺³ ions include, but are not limited to, DOTA and DPTA and derivatives thereof (see, Moi et al., J. Am. Chem. Soc. 110: 6266-6267 (1988)) and those chelators described in U.S. Pat. No. 4,885,363 and others, as outlined above.

When the paramagnetic ion is Dy⁺³, appropriate chelators will have 8 or fewer coordination atoms, since Dy⁺³ is capable of binding 8 or 9 coordination atoms. Suitable chelators are known in the art, as above.

Additionally, a manifold of routes for attaching chelating agents, crown ethers and cryptands to other molecules is available to those of skill in the art. See, for example, Meares et al., “Properties of In Vivo Chelate-Tagged Proteins and Polypeptides.” In, MODIFICATION OF PROTEINS: FOOD, NUTRITIONAL, AND PHARMACOLOGICAL ASPECTS;” Feeney, et al., Eds., American Chemical Society, Washington, D.C., 1982, pp. 370-387; Kasina et al., Bioconjugate Chem., 9: 108-117 (1998); Song et al., Bioconjugate Chem., 8: 249-255 (1997).

In addition, the complexes and metal ion complexes of the invention further comprise one or more targeting moieties. That is, a targeting moiety may be attached at any position, although in a preferred embodiment the targeting moiety does not replace a coordination atom. By “targeting moiety” herein is meant a group that serves to target or direct the complex to a particular location or association. Thus, for example, antibodies, cell surface receptor ligands and hormones, lipids, sugars and dextrans, alcohols, bile acids, fatty acids, amino acids, and peptides may all be attached to localize or target the contrast agent to a particular site.

An exemplary compound of the invention utilizes a glycosyl moiety, generally attached to the remainder of the BAMCA through a glycosidic linkage, as the relaxivity-modulating group. In an exemplary embodiment, the glycosyl moiety is conjugated to the linker arm via a glycosidic linkage, which is cleaved by a selected degradative enzyme, e.g., a glycosidase. Exemplary BAMCA according to this embodiment, in which the linker arm is an alkyl group, have the general formula: 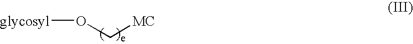
in which glycosyl-O is RM-X of Formula I; and e is an integer from 0 to 30.

Sugars of use in forming glycosides (i.e., glycosyl-O) are selected from any known naturally occurring sugars or their derivatives. Non-natural sugars and their derivatives are also of use as relaxivity-modulating groups. The selection of an appropriate glycosyl group is generally informed by the specificity of the desired enzyme target. The glycosyl groups can be mono- or oligo-saccharides. Exemplary sugars include Ara, arabinosyl; Fru, fructosyl; Fuc, fucosyl; Gal, galactosyl; GalNAc, N-acetylgalactosaminyl; Glc, glucosyl; GlcNAc, N-acetylglucosaminyl; Man, mannosyl; ManAc, mannosaminyl acetate; Xyl, xylosyl; NeuAc, and sialyl.

In an exemplary embodiment according to Formula III, the BAMCA includes a DO3A chelating agent. In a further exemplary embodiment, the DO3A complexes Gd⁺³.

In another exemplary embodiment, the invention provides a BAMCA having the structure: 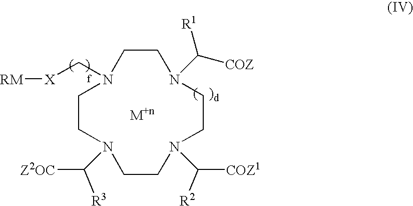
in which RM-X is as described for the compounds according to Formula I. The index “f” is an integer from 1 to 30; and the index “d” is 0, 1 or 2. As will be appreciated by those of skill in the art, the expansion or contraction of the ring represented by the index d can occur at any CH₂ position of the ring and is not limited to the position at which d is shown in Formula IV. M⁺ⁿ is a divalent or trivalent paramagnetic metal ion. R¹, R² and R³ are members independently selected from H, substituted or unsubstituted alkyl, substituted or unsubstituted heteroalkyl, substituted or unsubstituted aryl and substituted or unsubstituted heteroaryl. Z, Z¹ and Z² are members selected from OH, O⁻, and NR⁴R⁵. R⁴ and R⁵ are members independently selected from H, substituted or unsubstituted alkyl, substituted or unsubstituted heteroalkyl, substituted or unsubstituted aryl and substituted or unsubstituted heteroaryl.

In yet another exemplary embodiment, the invention provides a compound having the formula:

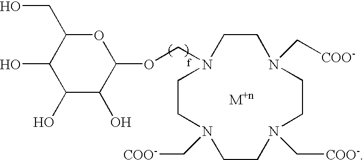

In a further embodiment, the glycosyl-O residue has the formula:

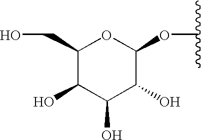

In another exemplary embodiment, the invention provides compounds having the formula: 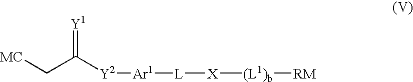
in which Y¹ and Y² are members independently selected from O, S and NH. Ar¹ is a substituted and unsubstituted aryl or substituted and unsubstituted heteroaryl moiety. The other symbols are discussed in the context of Formula I.

Exemplary compounds according to Formula V have the structure: 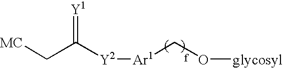
in which the index “a” represents and integer from 1 to 28.

Yet a further exemplary compound according to Formula V has the structure:

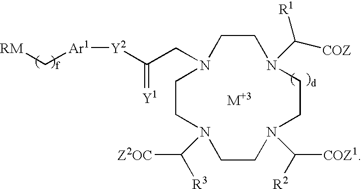

Another exemplary compound according to Formula I has the structure:

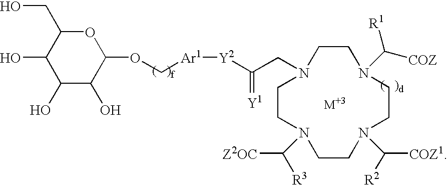

In a further exemplary embodiment, the compound of the invention has the formula:

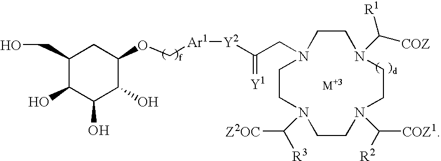

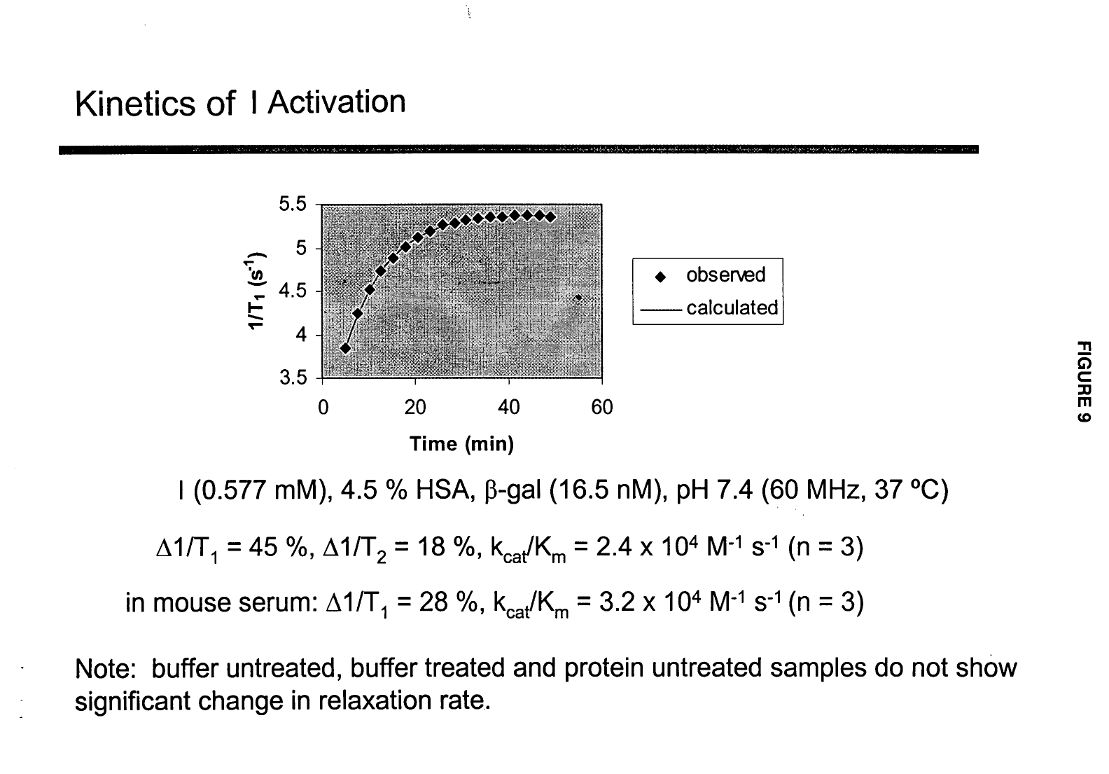

The compounds of the invention are also characterized by kinetics of activation that are rapid in comparison with art-recognized BAMCA. For example, an exemplary compound of the invention is activated on a time scale of seconds (FIG. 9), in contrast to EGAD. Thus, in an exemplary embodiment, an administered dose of a compound of the invention is at least 90% activated within less than 24 h, less than 12 h, less than 6 h, less than 3 h, less than 1 h, less than 30 min, less than 15 min or less than 5 minutes.

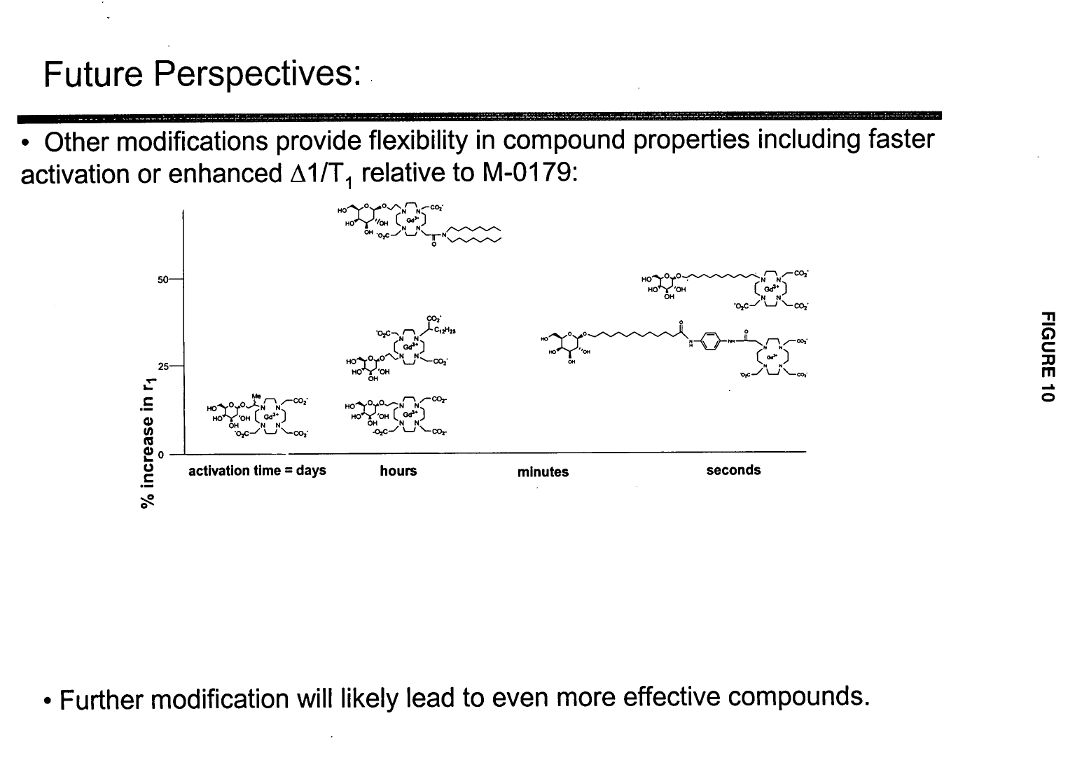

Additional compounds of the invention are set forth in FIG. 10, and they are presented in the context of their activation time scale. The art-recognized compound EGAD is also displayed for comparison.

In another aspect, the invention provides an activated BAMCA having the formula:
CM-L-X¹ (II)
in which X¹ is a residue formed by the cleavage of cleaveable bond X-L¹. The other symbols are as described for Formula I. The relaxivity of an activated compound according to Formula II is greater or less than that of an unactivated compound according to Formula I by an amount that is typically greater than about 15%, greater than about 20%, greater than about 25%, greater than about 30%, greater than about 35%, greater than about 40%, greater than about 45% or greater than about 50%.

In a preferred embodiment, the metal ion complexes of the present invention are water-soluble or soluble in aqueous solution; the inactivated complexes may be more water-soluble than their activated analogues or vice versa. By “soluble in aqueous solution” herein is meant that the compound of the invention in either its activated or inactivated form has appreciable solubility in aqueous solution and other physiologically-relevant buffers and solutions. Solubility may be measured in a variety of ways. In one embodiment, solubility is measured using the United States Pharmacopeia solubility classifications, with the metal ion complex being either very soluble (requiring less than one part of solvent for 1 part of solute), freely soluble (requiring one to ten parts solvent per 1 part solute), soluble (requiring ten to 10 thirty parts solvent per 1 part solute), sparingly soluble (requiring 30 to 100 parts solvent per 1 part solute), or slightly soluble (requiring 100-1000 parts solvent per 1 part solute).

Testing whether a particular metal ion complex is soluble in aqueous solution is routine, as will be appreciated by those in the art. For example, the parts of solvent required to solubilize a single part of MRI agent may be measured, or solubility in gm/ml may be determined.

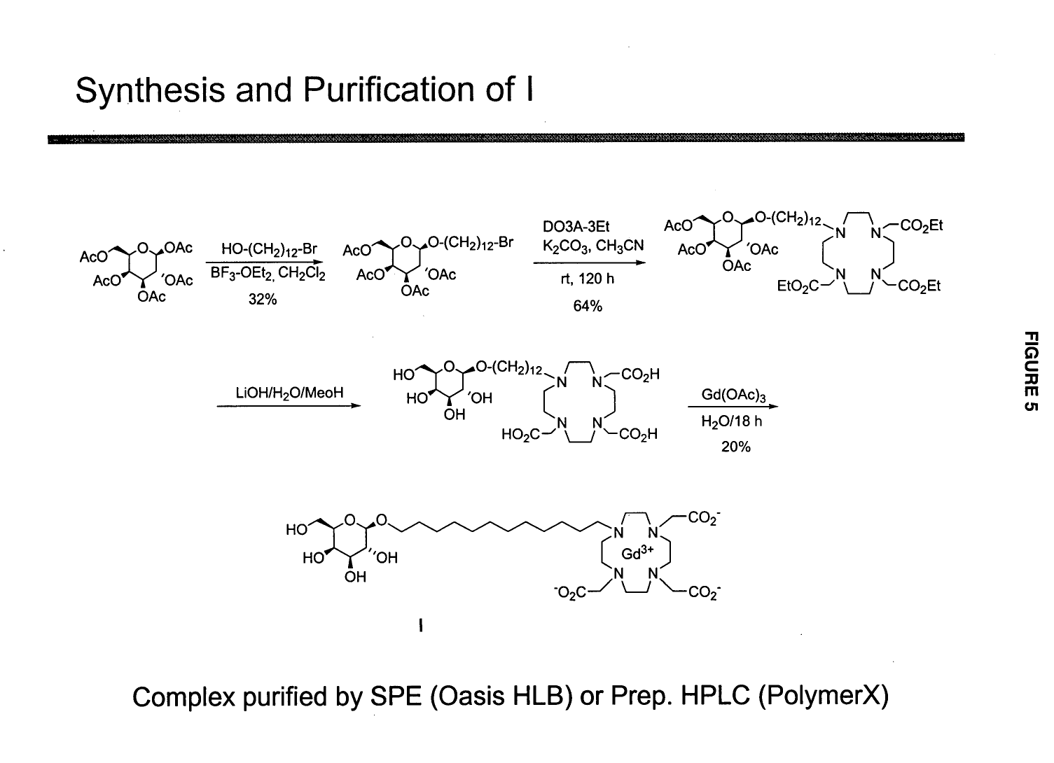

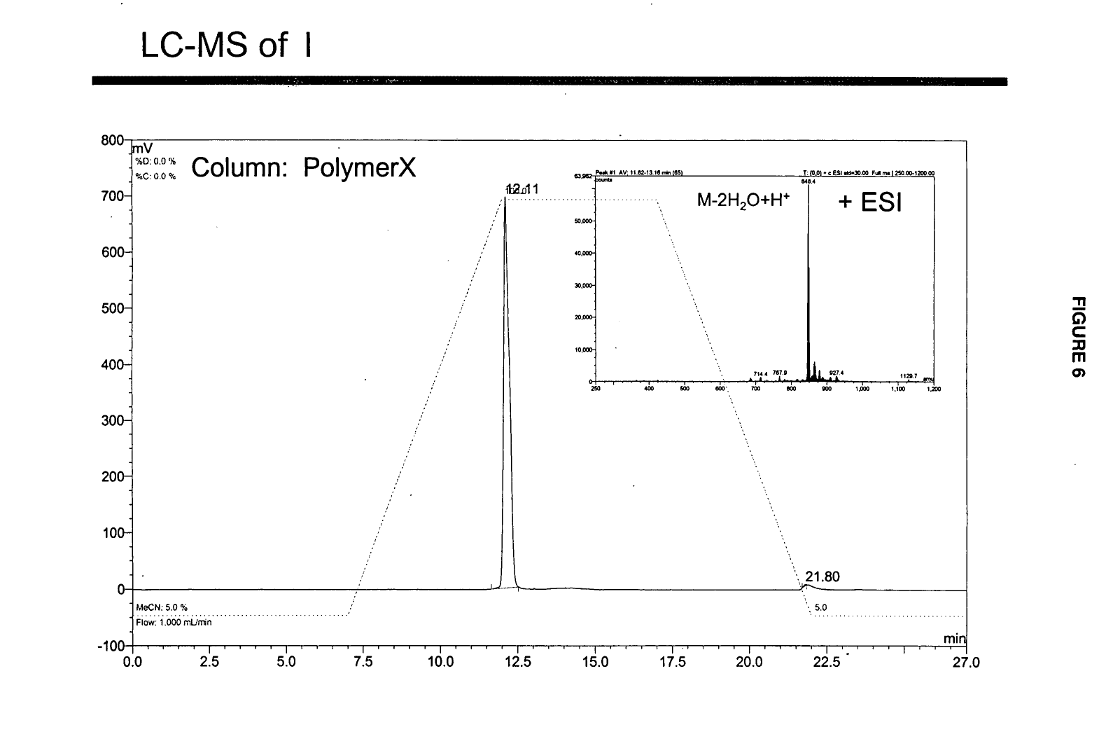

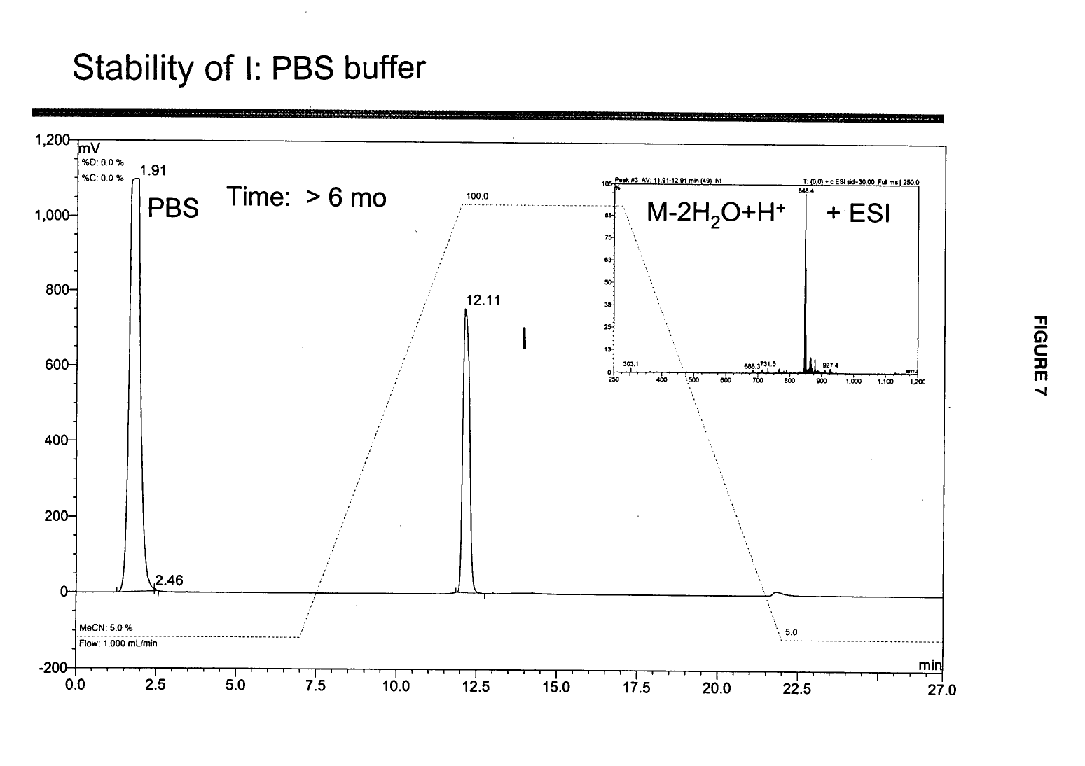

The complexes of the invention are generally synthesized using well-known techniques. An exemplary compound of the invention is prepared according to the scheme set forth in FIG. 5, which provides compounds of a high degree of purity (FIG. 6) that are stable to storage (FIG. 7)

For DOTA derivatives, the synthesis depends on whether nitrogen substitution or carbon substitution of the cyclen ring backbone is desired. For nitrogen substitution, such as is exemplified by the galactose-DOTA structures of the examples, the synthesis begins with cyclen or cyclen derivatives, as is well known in the art; see for example U.S. Pat. Nos. 4,885,363 and 5,358,704. For carbon substitution, well-known techniques are used.

The contrast agents of the invention are complexed with the appropriate metal ion as is known in the art. While the structures depicted herein all comprise a metal ion, it is to be understood that the contrast agents of the invention need not have a metal ion present initially. Metal ions can be added to water in the form of an oxide or in the form of a halide and treated with an equimolar amount of an unmetalated chelating agent. The chelating agent may be added as an aqueous solution or suspension. Dilute acid or base can be added if needed to maintain a neutral pH. Heating may be required.

The complexes of the invention can be isolated and purified by any art-recognized method, for example using HPLC systems (FIG. 6).

The relaxivity of the compounds is measurable by art-recognized methods. For example, the relaxivity of compound I of the invention was measured in the presence and absence of albumin (FIG. 8)

Pharmaceutical compositions comprising pharmaceutically acceptable salts of the contrast agents can also be prepared, e.g., by using a base to neutralize the complexes while they are still in solution. Some of the complexes are formally uncharged and do not need counterions.

Once synthesized, the metal ion complexes of the invention have use as magnetic resonance imaging contrast enhancement agents. Specifically, the functional MRI agents of the invention have several important uses. For example, they may be used to diagnose disease states. They may be used in real-time detection and differentiation of disease states. Moreover, they may be used in the identification and localization of toxin and drug binding sites. In addition, they may be used to perform rapid screens of the physiological response to drug therapy.

The metal ion complexes of the invention may be used in a similar manner to the known gadolinium MRI agents. See for example, Meyer et al., supra; U.S. Pat. No. 5,155,215; U.S. Pat. No. 5,087,440; Margerstadt et al., Magn. Reson. Med. 3:808 (1986); Runge et al., Radiology 166: 835 (1988); and Bousquet et al., Radiology 166: 693 (1988). The metal ion complexes are administered to a cell, tissue or subject as is known in the art. A “subject” for the purposes of the present invention includes both humans and other animals and organisms, such as experimental animals. In addition, the metal ion complexes of the invention may be used to image tissues or cells; for example, see Aguayo et al., Nature 322: 190 (1986).

Generally, sterile aqueous solutions of the contrast agent complexes of the invention are administered to a subject in one of a variety of ways, including orally, intrathecally and especially intraveneously in concentrations of from about 0.003 to about 1.0 molar, with dosages from 0.03, 0.05, 0.1, 0.2, and 0.3 millimoles per kilogram of body weight being preferred. Dosages may depend on the targets and the anatomical structures to be imaged. Dosages are also dependent on the relaxivity of the BAMCA; generally, the higher the relaxivity or the greater the change in relaxivity upon activation, the lower the dosage needed. Suitable dosage levels for similar complexes are outlined in U.S. Pat. Nos. 4,885,363 and 5,358,704.

In addition, the contrast agents of the invention may be delivered via specialized delivery systems, for example, within liposomes (see Navon, Magn. Reson. Med. 3: 876-880 (1986)) or microspheres, which may be selectively taken up by different organs (U.S. Pat. No. 5,155,215).

In some embodiments, it may be desirable to increase the blood clearance times (or half-life) of the MRI agents of the invention. This has been done, for example, by adding carbohydrate polymers to the chelator (U.S. Pat. No. 5,155,215). Thus, in one embodiment, the compounds of the invention include one or more polymeric moiety, e.g., polysaccharides, polyethers (e.g., poly(ethylene glycol), poly(propylene glycol) etc.) as substitutuents on the compositions of the invention. In an exemplary embodiment, one or more of MC, RM, L and/or L¹ in Formula I comprises a polymeric subunit. In yet another exemplary embodiment, one or more of R¹-R³ and/or Z-Z² of Formula IV comprises a polymeric subunit.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

1. A MRI agent comprising:
a) at least one paramagnetic metal ion bound to a metal complexing moiety in a stable, chelated form;
b) a relaxivity-modulating group, said group being capable of interacting with a target substance such that a relaxometric or physicochemical property of said agent are changed by said interacting; and
c) a linker arm comprising at least three atoms covalently linked to both said complexing agent and said relaxivity-modulating group.

2. The MRI agent according to claim 1 wherein said linker arm comprises at least five atoms.

3. The MRI agent according to claim 1 wherein said relaxivity-modulating group modulates said relaxivity essentially without interaction between said relaxivity-modulating group and an open coordination site of said paramagnetic metal ion.

4. The MRI agent according to claim 1 wherein cleavage of said relaxivity-modulating group from said agent forms an activated species that has a relaxivity greater or less than that of said agent.

5. The MRI agent according to claim 1 wherein said chelator is DOTA.

6. The MRI agent according to claim 1 wherein said chelator is DTPA.

7. The MRI agent according to claim 1 wherein said paramagnetic metal ion is selected from the group consisting of Gd(III), Fe(III), Mn(II), Yt(III), Cr(III) or Dy(III).

8. The MRI agent according to claim 1 wherein said relaxivity-modulating group comprises an enzyme substrate.

9. The MRI agent according to claim 1, wherein said change in relaxometric properties unpon interaction with said target substance is more than 20%.

10. The MRI agent according to claim 1 wherein said complexing moiety comprises a group that is selected from C₆-C₃₀ substituted or unsubstituted alkyl.

11. The MRI agent according to claim 1 wherein said complexing moiety comprises a poly(ethylene glycol) or methoxy-poly(ethylene glycol) moiety.

12. The MRI agent according to claim 1 wherein said relaxivity-modulating group comprises a group that is selected from C₆-C₃₀ substituted or unsubstituted alkyl.

13. The MRI agent according to claim 1 wherein said relaxivity-modulating group comprises a poly(ethylene glycol) or methoxy-poly(ethylene glycol) moiety.

14. A method of magnetic resonance imaging of a cell, tissue or patient comprising administering an MRI agent according to claim 1 to a cell, tissue or patient and rendering a magnetic resonance image of said cell, tissue or patient.

15. A MRI agent comprising:
a) a chelate of a paramagnetic metal ion;
b) a relaxivity-modulating group interacting with at least one coordination site of said paramagnetic metal ion essentially blocking coordination of water thereto; and
c) a linker arm comprising at least three atoms covalently linked to both said chelate and said relaxivity-modulating group, wherein
said relaxivity-modulating group is capable of being cleaved from said linker arm by a target substance such that the exchange of water in at least said one coordination site is increased.

16. The MRI agent according to claim 11, wherein cleaving said relaxivity-modulating agent increases relaxivity of said MRI agent by more than 20%.
